(19) United States
(12) Patent Application Publication
(10) Pub. No.: US 2006/0115521 A1
(43) Pub. Date: Jun. 1, 2006

(54) Materials, methods and apparatus for treating a body cavity

(75) Inventors: John Overton Hudson, Glenfield (GB); Alberto Bauer, Malaga (ES)

Correspondence Address: ARTHROCARE CORPORATION, 680 VAQUEROS AVENUE, SUNNYVALE, CA, 94085-3523, US

(73) Assignee: ArthroCare Corporation, Austin, TX (US)

(21) Appl. No.: 11/249,691

(22) Filed: Oct. 12, 2005

Related U.S. Application Data
(63) Continuation of application No. PCT/GB05/01874, filed on May 13, 2005.

(30) Foreign Application Priority Data
Oct. 6, 2004 (GB) 0422116.4

(51) Int. Cl. A61L 15/00; A61F 5/00

(52) U.S. Cl. 424/445; 602/1

(57) ABSTRACT

Materials, methods and apparatus for treating a body cavity. The present invention relates to materials, methods and apparatus for treating a body cavity or passageway. In particular, the invention relates to materials, methods and apparatus for treating a body cavity which has been subjected to some sort of trauma, such as surgery, resulting in a wound surface. The apparatus preferably comprises a two syringe sterilised package. One of the syringes contains CMC yarn which is converted to a CMC gel composition by mixing with water. The second syringe is used to measure water and mix it with the CMC yarn when connected to the yarn-containing syringe. When mixed, the CMC gel composition can be applied to a body cavity.

The present invention relates to materials, methods and apparatus for treating a body cavity or passageway. In particular, the invention relates to materials, methods and apparatus for treating a body cavity which has been subjected to some sort of trauma, such as surgery, resulting in a wound surface.

There are numerous reasons why a patient's mucous membrane might have suffered trauma and benefit from such treatment. For example, the patient may have epistaxis (intra nasal bleeding). Alternatively, the patient may have undergone surgery of the nasal cavity (such as rhinoplasty, septoplasty, turbinoplasty or nasal sinus surgery, e.g. treatment of polyps in the nasal and/or sinus).

The term “nasal cavity” as used herein includes within its meaning not only the nasal cavity, including the lower, middle and upper nasal meatus and the nasal pharynx, but all other connected cavities having a mucosal membrane. Thus included within the meaning of this term is the paranasal sinuses such as the maxillary sinus (a pyramidal bilateral chamber, which is bordered inferiorly by the alveolar ridge and palate, medially by the nasal cavity, and laterally anteriorly by the cheek), the ethmoidal cell complex (bilateral air cells between the medial wall of the orbit and the lateral wall of the nose), the sphenoid sinus (a bilateral deep midline cavity), and the frontal sinus (a bilateral bony chamber, which is bordered inferiorly by the roof of the orbit, anteriorly by the skin of the forehead and posteriorly by the anterior cranial fassa).

Nasal plugging devices and, more generally, haemostatic devices for other purposes, are well known in the art for the treatment of bleeding inside body cavities and vessels (see, for example, WO 02/47558, WO 01/85037, WO0185036 and WO9857586, the disclosures of which are incorporated herein by reference). The aim of their use is the prevention of bleeding, that is, haemostasis, and for post operative packing to control bleeding and promote healing. Various haemostatic and packing devices are known including sponge materials which expand when wetted and balloon devices which can be expanded hydraulically or pneumatically, and can apply pressure to the source of the bleeding.

The present invention provides further materials, methods and apparatus for treating a body cavity. Although the invention is particularly described with reference to the nasal cavity, it is to be understood that the various aspects of the invention will be of benefit to the treatment to other body cavities, particularly the ear and throat (tonsils).

According to a first aspect, the invention provides apparatus for treating a wound surface in a body cavity comprising a dispenser and characterised in that the dispenser contains a carboxymethylcellulose (CMC) gel-forming fibre or yarn.

The production of CMC and the knitting of fabrics therefrom is described in greater detail in WO 01/23653 and WO 02/47558, the disclosures of which are incorporated herein by reference. CMC may be made by the chemical conversion of a variety of cellulosic materials, such as viscose rayon, cotton, etc. One cellulosic yarn suitable for the present invention is a Lyocell yarn. It is available from Spinneroff Streif AG, Zurichstrasse 170, Uathal, Switzerland. Lyocell is a solvent spun cellulose, produced from the natural cellulose in wood pulp by dissolution of the pulp in a solvent and then extruding the solution through a multiple-hole die, called a spinneret, to form a yarn comprised of a plurality of continuous strands. The solvent is vaporized in the process, leaving a continuous multi-filament yarn composed of pure cellulose.

The filaments in such a yarn may be chopped into staple form and spun into a yarn in a way similar to that used in processing cotton fibre.

In the conversion of cellulose to sodium carboxymethylcellulose, less than all of the cellulose building blocks may be converted to the sodium carboxymethylcellulose form and the degree of this conversion will dictate the degree to which a resultant CMC yarn will absorb water and form a gel therewith.

A preferred CMC is the active ingredient in “Gel Knit™” which is used in the “Rapid Rhino™” range of epistaxis and nasal dressings.

CMC gels upon contact with water, blood or body fluids, and swells to absorb such materials. CMC also facilitates blood clotting while absorbing any exudate and is, therefore, haemostatic. By “haemostatic” we mean the ability to arrest, stem or prevent bleeding by means other than induced tissue growth. Preferably, the haemostatic action includes vasoconstriction and/or blood coagulation.

In addition, it is well known that CMC is hydroscopic so it does not readily dry into clotted blood, and therefore can be removed easily without causing re-bleeding. If it does dry, it can be easily re-gelled by wetting with water. The above mentioned properties of CMC are conducive to the formation of a wound-healing microatmosphere when the CMC gel is used to treat a body cavity.

Preferably the gel-forming fibres of the invention are formed into a yarn.

The word “yarn” as used herein, refers to an indefinite length of material suitable for weaving, knitting or braiding, typically comprised of one or more continuous strands of material or a multiplicity of relatively short length fibres spun into a fibre bundle of indefinite length, or some combination of continuous strands and spun fibres.

Preferably, the yarn is formed as a “soft twist” (sometimes also called “hosiery twist”), which is a yarn with less than approximately 1200 turns per metre. For the purposes of the present invention CMC yarn having from approximately 700 to approximately 900, and especially approximately 850 turns per metre, has been found to be particularly useful.

Provision of the CMC gel-forming fibres as a yarn has been found to confer unexpected benefits in terms of the ease with which the CMC can be hydrated with water or a water mixture. CMC yarn is considerably easier to hydrate to form a gel composition than CMC fibres or CMC powder. Also, a continuous length of CMC yarn is easier to hydrate than a corresponding length of CMC yarn which has been chopped into a plurality of shorter pieces. Again this is quite an unexpected finding.

Combinations of different gel-forming CMC fibres or yarns may be used within the scope of the present invention. Such combinations may be made by forming a yarn from different gel-forming fibres and/or by knitting combinations of different gel-forming yarns.

Conveniently; the gel-forming fibres of CMC may be provided as a single CMC type or as a mixture of different CMC fibre types, each type having a different degree of solubility which can range from high to insoluble.

The “solubility” of a CMC fibre or yarn as referred to herein can be determined according to the following solubility test.

A weighed amount (“starting weight”) of CMC fibre or yarn is added to an excess of water under stirring. The solution so formed is then filtered to separate any solid or gelled material that has not dissolved.

Water is then evaporated off the filtrate to leave a solid residue. The solid residue is weighed and the residue weight is deducted from the starting weight to give the amount of CMC which has been dissolved. For example, if 100 g CMC is the starting weight and 2 g is the final residue weight, the degree of solubility will be 100−2=98%.

As referred to herein “High” solubility CMC means 95% or more; “low” solubility CMC means less than 50%, and “insoluble” CMC means less than 10% of the starting weight is lost in the solubility test described above.

By way of example, low solubility CMC gel compositions of the invention typically have a permanency/residence time in the nasal cavity of from 8 to 15 days following treatment whereas a permanency/residence time of from 4 to 8 days is typical with a high solubility CMC gel composition.

A skilled person can control the permanency/residence time of the gel composition in the body cavity according to the invention by selecting gel-forming CMC fibres or yarn having an appropriate solubility. For example, a gel composition formed from high solubility fibres or yarn will readily absorb body fluids in use and eventually lose its physical integrity and be “washed away” or dissolved from the body cavity without medical intervention. By contrast, increasing the proportion of low solubility and/or insoluble fibres or yarn will serve to increase the permanency/residence time of the gel composition in the body cavity.

The stiffness or viscosity of the gel composition can also be controlled by varying the amount of water or an aqueous mixture (solution, dispersion, etc) that is mixed with the gel-forming fibre or yarn to make the gel composition.

Preferably, the gel composition of the invention is made by mixing 1 part by weight of CMC fibre or yarn with from 4 to 12 parts by weight of water (or water mixture). For example, 1 gram of CMC yarn is mixed with from 4 to 12 grams of water. Of course, in view of the density of water a volumetric measurement is possible, with 4 to 12 grams corresponding to 4 to 12 mls. For most aqueous mixtures an approximate volumetric measurement is also suitable, as will be appreciated by a skilled reader.

The above ratio of CMC fibre or yarn to water provides a gel composition with a viscosity range which is suitable for treating body cavities.

Still further, the composition may be reinforced by the inclusion of non-gel-forming fibres.

In general, it is preferred that the CMC gel compositions of the invention have a viscosity of from 1×10⁵ centipoise up to 3.0×10⁶ centipoise as determined by the falling sphere test at 20° C. and atmospheric pressure.

A convenient test method for determining whether a CMC gel composition has a viscosity suitable for use to treat a body cavity according to the invention can be described as follows:

A sample of the gel to be tested is placed into a vertical cylinder such as a glass graduated cylinder.

A steel sphere is then dropped into the gel and the time taken for it to drop a measured distance is noted.

The viscosity may be calculated as follows:
2 (dp) g a²/9 v

Where:

dp (delta p) is the difference in density between the sphere and the gel.

g is the acceleration due to gravity (numerically=9.81)

a is the radius of the sphere.

v is the velocity of the falling sphere.

SI units for the results will be expressed in Pascal seconds (Pa s). To convert Pa s to the more commonly used centipoises (Cp) simply multiply by 1,000.

Skilled persons will appreciate that many alternative and/or modified apparatus arrangements are possible within the scope of the present invention. For example, although a syringe is preferred, the dispenser may be provided in any other suitable form, such as a flexible bag or tube.

For example, a flexible plastic bag may be provided which comprises a CMC fibre or yarn compartment and a water compartment separated by a breakable membrane. The membrane is broken to permit mixing of the CMC fibre or yarn and water to form the gel composition. Conveniently, the bag is provided with an outlet point through which the gel composition can be dispensed. The outlet port may simply be a corner or neck of the bag which is cut off to make a dispenser which resembles an icing bag.

Preferably, however the outlet port comprises a connector to permit connection to a syringe or an applicator for applying the gel composition to a body cavity.

Although termed a “dispenser” it will be appreciated that the container for the gel-forming CMC fibre or yarn of the invention is not necessarily the container from which the gel composition is dispensed to treat a body cavity. The dispenser may simply serve as a container from which the gel composition is transferred to a further container, such as a syringe, from which the gel composition is dispensed in use to treat a body cavity. Such an arrangement is described in Example 1 below.

The apparatus is preferably provided with a container and a connection means which permits fluid communication between the dispenser and container.

The dispenser and/or container is preferably a syringe and the connection means can be any means of connecting the syringe nozzles together so that water or an aqueous mixture within one syringe can be mixed with the gel-forming CMC fibre or yarn in the other to form a gel composition of the invention.

Conveniently, one or both of the syringes is graduated to permit measurement of the requisite amount of gel-forming fibre and/or water or an aqueous mixture.

The water mixture can be an aqueous solution or dispersion of a medicament.

According to a second aspect of the invention there is provided a gel composition for treating a body cavity characterised in that the gel composition is formed by mixing from approximately 4 to 12 parts by weight of water or water mixture with from approximately 0.5 to 3 parts by weight of a CMC gel-forming fibre or yarn.

More preferably, the ratio of water or water mixture to CMC fibre or yarn is from 5 to 8 parts of water or water mixture per part by weight of CMC fibre or yarn.

Various optional ingredients can also be included in the final hemostatic gel composition such as preservatives and small amounts of pharmacologically active ingredients referred to herein as (“medicaments”). For example an antibiotic or antimicrobial agent such as metronidazole, silver sulphadiazine, neomycin or penicillin and antiseptic agent such as povidone iodine and anti-inflammatory agent such as hydrocortisone or triamcinolone acteonide or a skin protective agent such as a zinc oxide can be included.

Any anti-inflammatory compound may be used by the present invention. It may be a steroid or a non-steroid. Preferred anti-inflammatory compounds as described above are selected from the group consisting of cortisone (e.g. Prednisolene, Betamethasone, fluticasone, (Flutide™), fluocortine (Lenen™), flunisolid (Santaris™), Lenen™ (a water soluble powder)) cortisol (e.g. Hydrocortison Hoechst™) or biologically active derivatives of either. An example of a biologically active derivative is cortisone acetate (USP) (an ester of cortisone), which is used as an anti-inflammatory and immunosuppressant in a wide variety of disorders. Other derivatives are also included. A “derivative” in this context refers to a modified compound which retains the basic properties of the compound from which it was derived. For example anti-inflammatory activity, effects on wound healing, immunosuppression activity, levels of toxicity and stability have not been significantly changed.

According to the invention there is also provided a component for use together with the apparatus and/or compositions of the inventions comprising a sterile container containing a sterile composition, the composition comprising an anti-inflammatory compound in the form of an aqueous solution or in a form that is dissolvable in water, at a concentration or in an amount that is effective to reduce inflammation of a mucous membrane in a patient's body cavity or suitable to be dissolved, or for dilution, to a concentration that is effective to reduce inflammation of a mucous membrane in a patient's nasal cavity.

The skilled person is able to determine an effective concentration of any given anti-inflammatory compound by routine testing.

For example, Prednisolone (a cortisone product), has a 4 times relative glucocorticoid potency, whereas Betamethasone, the most common topical cortisone for the nose, has 30 times relative glucocorticoid potency. When used in aqueous solution at a concentration suitable for delivering a 0.05 mg dose, a single dose of Betamethasone is normally given postoperatively up to 4'2 single doses per day, which is the equivalent of 0.4×30/4=3 mg Prednisolone. A typical effective concentration of cortisone that may be used with the present invention may be around 10-40 mg Prednisolone per 1 ml.

Alternatively, the composition of the anti-inflammatory compound can be provided in a concentrated aqueous form or powdered form, which allows the individual practitioner to dissolve/dilute the powder or solution to a concentration appropriate for the patient in hand, given the age, sex, weight and condition of the patient and the identity of the cavity being treated. For example, an aqueous solution of the anti-inflammatory compound can be provided in a form that requires dilution 2-fold, 5-fold, 10-fold, 20-fold, 50-fold, 10²-fold, 10³-fold, 10⁴-fold, 10⁵-fold, 10⁶-fold, 10⁷-fold, 10⁸-fold, or more to provide a therapeutically appropriate concentration.

The present invention also contemplates the delivery of compounds other than anti-inflammatory drugs. Other drugs considered include:—antiphogistic drugs, steroids, non-steroids, anti-histamerics, anticholinergics, antibiotics, antimycotics and botulinum toxin; the latter of which may be used as an anti-allergic drug for allergic rhinitis therapy. It will, therefore, be appreciated by the reader that reference in this application to “anti-inflammatory compound” and the like can, for the more general practice of this invention, also be read as including other compounds, such as those listed above.

Preferably the apparatus and/or gel compositions of the invention are provided in the form of a package. The package is preferably sterilised and sealed. Sterilisation may be accomplished by a variety of methods, such as exposure to ethylene oxide.

In this process the goods are placed into an airtight chamber. After spending some time to ensure that the temperature and humidity are at optimum conditions, a vacuum is drawn on the chamber. Ethylene oxide gas is introduced. This remains in the chamber for some time in order to destroy any bacteria.

The gas is then extracted and a series of vacuum and pressure cycles are given to purge all residual gas from the system.

As is explained below, the packaging is such that the gas is allowed to penetrate but bacteria are not.

Examples which embody preferred aspects of the invention will now be described with reference to the accompanying figures in which:

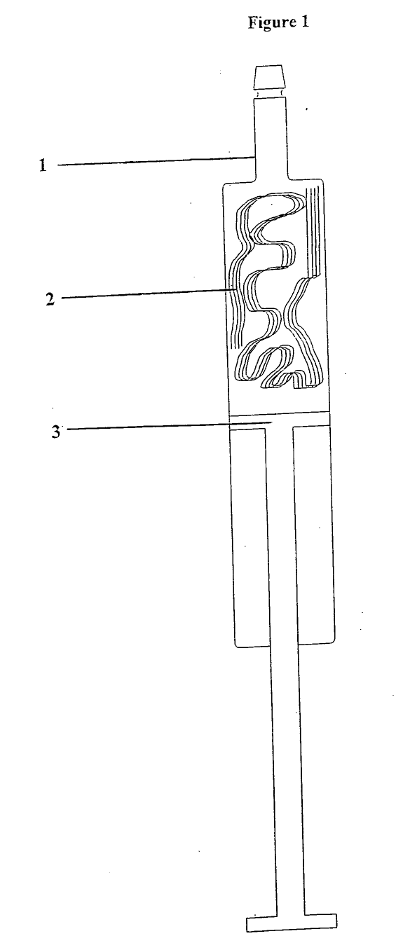

FIG. 1 shows a syringe dispenser (1) of the invention containing CMC yarn (2). The yarn is arranged loosely within the body of the syringe and the piston (3) is in its semi-retracted position to permit uptake of water to hydrate the CMC yarn and form the CMC gel composition.

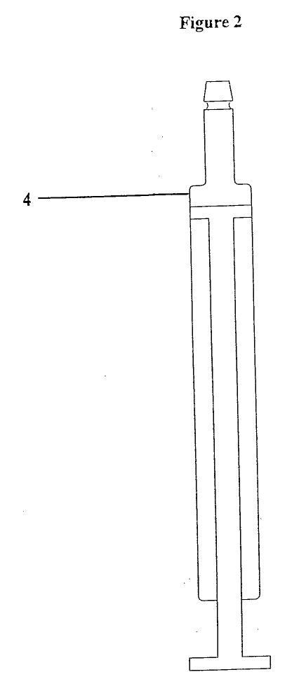

FIG. 2 shows a second smaller syringe (4) container of the invention. It is graduated to permit measurement and metering of water or a water mixture into the syringe dispenser shown in FIG. 1.

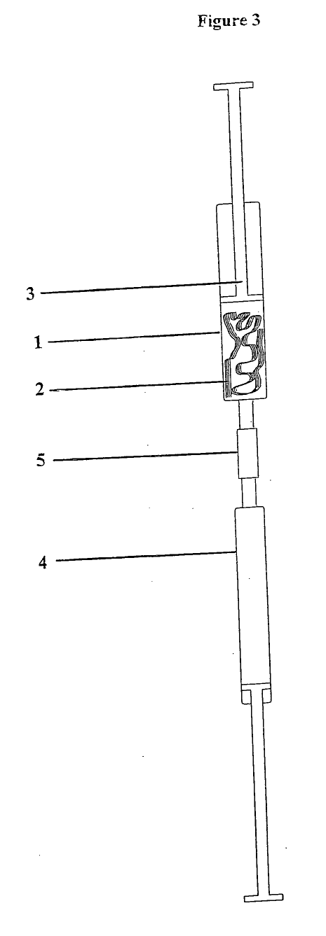

FIG. 3 shows the syringes of FIGS. 1 and 2 connected by a connector (5) which permits fluid communication between the two syringes.

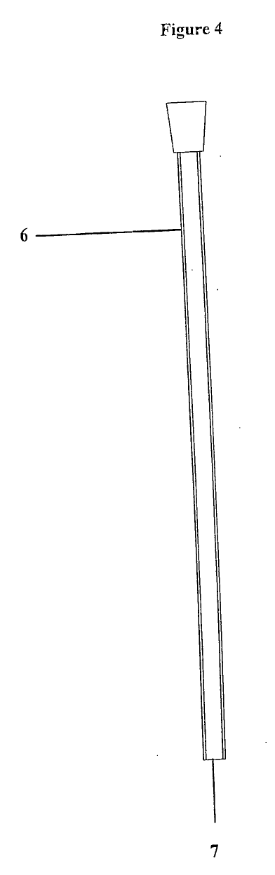

FIG. 4 shows an applicator (6) of the invention which includes an adaptor at its proximal end for fitting to the nozzle of one of the syringes of FIG. 3.

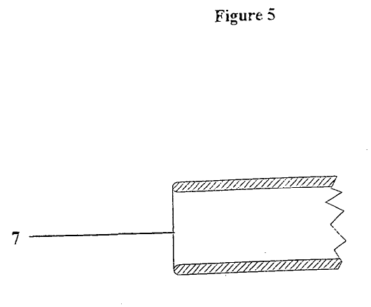

FIG. 5 shows the distal end (7) of the applicator. The “tipped” profile of the distal end causes the least possible disturbance to the delicate mucous membranes which line body cavities such as the nose.

### EXAMPLE 1

### Kit Comprising Dispenser and Gel-Forming Fibre in Dry Form

In this embodiment apparatus for treating a body cavity comprises a large (22 mm diameter) syringe dispenser (1) containing a gel-forming fibre (2) consisting of 15 decitex (dtex) viscose spun yarn converted to CMC, as shown in FIG. 1. The CMC yarn (1 gram) is packed loosely inside the syringe body with the piston (3) in a semi-retracted position so as not to compress the yarn inside the syringe body, whilst permitting uptake of water for mixing to form the gel composition. Packing the CMC yarn loosely in this manner facilitates easy hydration of the yarn to form a gel composition.

As shown in FIG. 2, the apparatus further comprises a container in the form of a second smaller (10 mm diameter) syringe (4) which is graduated to permit metering of a precise amount of water or water mixture (e.g. 5 to 8 mls).

The water or water mixture may be contained within the second syringe (4) or provided separately.

The preferred apparatus includes a connector (5), shown in FIG. 3, which permits connection of the nozzle of the syringes and fluid communication therebetween, as shown in FIG. 3.

The preferred apparatus also includes an applicator (6) in the form of an elongate tubular member which is tipped at its distal end (7), as shown in FIGS. 4 and 5. The dimensions of the applicator are selected to permit application of a gel composition into a selected body cavity. For nasal applications the outside diameter of the applicator is preferably from 3 to 5.5 mm; the internal diameter is 2-5 mm and the length is from 80 to 140 mm.

The above apparatus is packaged and sterilised.

A typical package method is to use a thermoformed polyester blister with a tyvek™ top seal. The blister is formed so that all the individual components of the kit have their own compartment.

Tyvek™ is a proprietary semi permeable non woven polyester membrane. It is constructed such that sterilising gas is allowed through the pores in the membrane but bacteria are not allowed to pass through.

The kit may thus be packed into the seal blister/tyvek™ membrane and the contents sterilised by exposure to sterilising gas. Such a gas would typically be ethylene oxide. For sterilisation, the syringe is open, that is, any cap is left off so that the gas can come into contact with the CMC yarn.

In order to treat a body cavity the apparatus of the invention may be used as follows:
1. Measure an appropriate amount of water into the second syringe (4).
2. Connecting the two syringes together with the aid of a connector (5).
3. Holding the two syringes vertical with the water containing syringe (4) uppermost.
4. Quickly transferring the water into the fibre containing syringe (1).
5. Allowing the water to soak into the fibres—aid by gentle shaking.
6. Disconnecting the syringes and expelling excess air from the fibre containing syringe.
7. Re-connecting the two syringes with the second syringe-closed.
8. Transferring the gel from syringe to syringe to mix the gel and finally collecting the gel (approximately 5 to 10 mls.) in one syringe (usually the smaller syringe).
9. Removing the empty syringe and connector.
10. Attaching the applicator (6) to the gel-filled syringe.
11. Applying the gel to the body cavity to be treated via the tipped distal end (7) of the applicator.

### EXAMPLE 2

### Kit Comprising Dispenser with Pre-Mixed CMC Gel Composition

In this embodiment the dispenser is provided with a pre mixed CMC gel composition.

The preferred gel composition is formed as follows:

The CMC fibre and water are mixed in large quantities using standard mixing machinery common in the food and drug industries.

However, the only systems of sterilisation available to liquids or gels are those that involve radiation. (Usually gamma or X-Ray). Such radiation causes chemical degradation to CMC.

It is therefore necessary to sterilise the components of the mixture and then mix and package under sterile conditions.

After mixing, the syringes may be filled using a pressure system. The commercial process of filling syringes is well known to those in the trade.

1-41. (canceled)

42. An apparatus for treating a wound surface in a body cavity comprising:
a dispenser, said dispenser comprising a CMC gel-forming fiber.

43. The apparatus as recited in claim 42 further comprising a container, fluidly connectable to said dispenser such that liquids may transferred to and from the dispenser.

44. The apparatus as recited in claim 43 wherein said container comprises water or a water mixture for treating the gel-forming fiber to form a haemostatic gel composition.

45. The apparatus as recited in claim 44 wherein the water mixture is an aqueous solution and/or dispersion of a medicament.

46. The apparatus as recited in claim 42 wherein the dispenser contains a predetermined amount of gel-forming fiber.

47. The apparatus or a method as recited in claim 46 wherein the predetermined amount is from 0.5 to 3.0 grams.

48. The apparatus as recited in claim 42 wherein the gel-forming fiber comprises a mixture of two or more fibers having a different solubility.

49. The apparatus as recited in claim 42 wherein the gel-forming fiber is provided together with a reinforcing fiber.

50. The apparatus as recited in claim 42 wherein the fibre or fibres is provided in the form of a yarn.

51. The apparatus as recited in claim 50 wherein the yarn is formed as a “soft twist” having from approximately 700 to approximately 900 turns per metre.

52. The apparatus as recited in claim 43 further comprising a detachable applicator for applying gel to a body cavity.

53. A biocompatible CMC gel composition for applying to a body cavity comprising:
a gel formed by mixing from approximately 4 to 12 parts by weight of water and/or water mixture with from approximately 0.5 to 3 parts by weight of gel-forming CMC fiber and/or yarn.

54. The gel composition as recited in claim 53 further comprising a medicament for treating a wound.

55. The gel composition as recited in claim 54 wherein the medicament is selected from one or more anti-inflammatory and/or anti-microbial agents.

56. A method for treating wounds comprising:
providing a dispenser holding a CMC gel forming fiber;
forming a gel within the dispenser; and
injecting the gel from the dispenser into a target site.

57. The method of claim 56 further comprising providing a container holding a liquid to be mixed with said gel forming fiber.

58. The method of claim 57 comprising connecting said container and said dispenser to each other and injecting the liquid into said dispenser to form said gel.

59. The method of claim 58 comprising transferring said gel from the dispenser to the container and back to said dispenser.

60. The method of claim 56 wherein said target site is the nasal cavity.

61. The method of claim 56 wherein said gel forming fiber includes only one elongate CMC fiber.
